(19) United States
(12) Patent Application Publication
(10) Pub. No.: US 2009/0252725 A1
(43) Pub. Date: Oct. 8, 2009

(54) Use of CD23 Antibodies to Treat Malignancies in Patients with Poor Prognosis

(75) Inventors: Sarah Harris, San Diego, CA (US); Shabnam Tangri, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec MA Inc.

(21) Appl. No.: 12/400,695

(22) Filed: Mar. 9, 2009

Related U.S. Application Data
(60) Provisional application No. 61/034,901, filed on Mar. 7, 2008.

(51) Int. Cl. A61K 39/395; C12N 5/06

(52) U.S. Cl. 424/133.1; 424/172.1; 435/375

(57) ABSTRACT

The invention relates to methods of treating B-cell chronic lymphocytic leukemia, and other CD23⁺ malignancies, in patients with poor prognostic markers. The method comprises administration of an CD23 antibody, including for example, lumiliximab to a mammal that over expresses a poor prognostic marker. The method can also comprise administration of lumiliximab in combination with fludarabine, cyclophosphamide and rituximab. Patients with poor prognostic markers include, for example, patients that overexpress ZAP70, CD38, β2-microglobulin and/or soluble CD23.

### CROSS-REFERENCE TO RELATED APPLICATIONS

This application claims the benefit of U.S. Prov. Appl. No. 61/034,901, filed Mar. 7, 2008, which is herein incorporated by reference in its entirety.

### BACKGROUND OF THE INVENTION

1. Field of the Invention

The invention relates to methods of treating chronic lymphocytic leukemia (CLL or B-CLL) and other CD23⁺ malignancies using anti-CD23 antibodies. In particular, the invention relates to the treatment of such malignancies in patients with poor prognostic markers including high ZAP70 and/or CD38 expression levels.

2. Background Art

Many advances in cancer treatment have been made over the past decade, including the development of multiple tumor-specific antibodies which have been approved for cancer therapy. However, because cancer is such a heterogeneous disease, treatments typically work in only a subset of cancer patients. More recent studies have also helped to identify a number of prognostic markers that are associated with rapid disease progression or short survival. For example, several markers have been associated with poor prognosis in chronic lymphocytic leukemia.

Chronic lymphocytic leukemia (CLL or B-CLL) is one of four major types of leukemias, or cancers of the blood and marrow. It involves the overpopulation of clonal lymphocytes (CD5⁺/CD19⁺ cells) that morphologically resemble mature B cells.

CLL is believed to be due to a inhibition of apoptosis which leads to cell accumulation. Expression of CD23 protein has been found to be higher in CLL patients. CD23 is a low affinity IgE receptor that is expressed in cells of several hematopoietic lineages. It is a member of the lectin family of calcium-dependent Type II integral membrane proteins, but exists as both a membrane bound form and a soluble form. The membrane form includes an intracellular domain, a transmembrane domain, an α-helical coiled coil that is thought to be important for oligomerization during ligand binding and an extracellular lectin binding domain. It is expressed on B cells, monocytes, macrophages, eosinophils, platelets and dendritic cells. It is thought to be important for inhibition of IgE synthesis and inhibition of B cell growth and differentiation. Increased levels of membrane CD23 are seen on CLL cells.

Soluble CD23 may be formed by protease cleavage of membrane bound CD23 or may be released from intracellular stores. Soluble CD23 proteins ranging in size from less than 18 kD to 45 kD have been identified, and a 25 kD fragment has been shown to be stable and functional. The functions include upregulation of IgE synthesis, simulation of B cell growth and differentiation and inhibition of B cell apoptosis. Soluble CD23 is found at low levels in healthy individuals and increased levels have been observed in CLL patients.

The fact that CD23 is expressed on CLL cells and plays a role in regulating B cell growth and differentiation makes it an attractive therapeutic target, and CD23 antibodies have been shown to induce apoptosis, as measured by caspase-3 activity, in CD23⁺ tumor cells and to increase survival in a human B-lymphoma/SCID mouse model (See for example, U.S. Patent Application Publication No. 2006/0171950, which is herein incorporated by reference in its entirety).

In addition to CD23 antibodies, several other CLL treatment methods have been suggested including chemotherapeutic molecules, such as fludarabine and cyclophosphamide, as well as other antibodies such as rituximab (an anti-CD20 antibody), and many have been shown efficacy in at least a subset of patients. However, several markers of poor prognosis, including high levels of ZAP70, CD38, β2-microglobulin and soluble CD32, have been identified for CLL patients and the available treatments are not thought to be effective for patients with poor prognostic markers. Therefore, a need to identify methods of treating patients with poor prognostic markers still exists.

### BRIEF SUMMARY OF THE INVENTION

This invention is based on the surprising discovery that CD23 antibodies can induce apoptosis in cells expressing poor prognostic markers and that CD23 antibodies are therapeutically effective in treating patients with poor prognostic markers.

In some embodiments of the invention, the invention provides methods of treating a CD23⁺ malignancy in a mammal in need thereof comprising administering to said mammal a therapeutic amount of a CD23 antibody or fragment thereof, wherein the mammal over expresses a poor prognostic marker. In other embodiments, the invention provides methods of treating a leukemia in a mammal in need thereof comprising administering to said mammal a therapeutic amount of a CD23 antibody or fragment thereof, wherein the mammal over expresses a poor prognostic marker.

In some embodiments, the invention provides methods of treating a CD23⁺ malignancy or a leukemia in a mammal in need thereof comprising administering to said mammal a therapeutically effective amount of a CD23 antibody or fragment thereof, wherein the mammal has been demonstrated to overexpress said poor prognostic marker. In further embodiments, the methods of the invention further comprises determining whether said mammal over expresses said poor prognostic marker.

In some embodiments, the invention also provides methods of inducing apoptosis in a cell expressing a poor prognostic marker comprising contacting the cell with a CD23 antibody.

In some embodiments of the invention, the poor prognostic marker is selected from the group consisting of ZAP70, CD38, β2-microglobulin and soluble CD23.

In some embodiments of the invention the CD23 antibody is lumiliximab, an antigen binding fragment thereof, an antibody that competitively inhibits binding of lumiliximab to CD23 or an antigen binding fragment thereof. In some embodiments of the invention, the CD23 antibody is lumiliximab.

In some embodiments the administration of CD23 antibody results in increased caspase-3 activity. In some embodiments of the invention, administration of CD23 antibody results in increased apoptosis.

In some embodiments the invention provides for methods of treating a CD23⁺ malignancy or a leukemia in a human in need thereof comprising administering to said human a therapeutically effective amount of a CD23 antibody or fragment thereof, wherein the human over expresses said poor prognostic marker.

In some embodiments of the invention, the administration of the CD23 antibody is achieved by oral administration, nasal administration, parenteral administration, transdermal administration, topical administration, intraocular administration, intrabronchial administration, intraperitoneal administration, intravenous administration, subcutaneous administration, intramuscular administration, buccal administration, sublingual administration, vaginal administration, by inhalation, by an implanted pump, and a combination of two or more thereof. In some embodiments, the administration is intravenous.

In some embodiment of the invention, the CD23 antibody is used in conjunction with an additional pharmaceutical compound effective for treating, preventing or inhibiting a malignancy. In some embodiments, the pharmaceutical compound is selected from the group consisting of therapeutic antibodies, immunosuprresive agents, cytotoxic agents, chemotherapeutic agents and/or cytokines. In some embodiments of the invention, the CD23 antibody is used in conjunction with fludarabine, cyclophosphamide and rituximab.

In some embodiments the CD23⁺ malignancy is selected from the group consisting of relapsed Hodgkin's disease, resistant Hodgkin's disease high grade, low grade and intermediate grade non-Hodgkin's lymphoma, B cell chronic lymphocytic leukemia (B-CLL OR CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphoma, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkitt's and non-Burkitt's; follicular, predominantly large cell, follicular, predominantly small cleaved cell; and follicular, mixed small cleaved and large cell lymphomas. In some embodiments the CD23⁺ malignancy is B cell chronic lymphocytic leukemia (CLL). In some embodiments of the invention, the leukemia is B cell chronic lymphocytic leukemia (CLL).

In some embodiments of the invention, the poor prognostic marker is ZAP70 or CD38 and the poor prognostic marker is expressed on 30% or more of the CLL cells of the mammal.

### BRIEF DESCRIPTION OF THE DRAWINGS/FIGURES

FIG. 1: Flow cytometery scheme to assess expression of prognostic markers on peripheral blood CLL cells isolated from patients being treated with Lumiliximab in combination with fludarabine, cyclophosphamide and rituximab (L+FCR). Lymphocytes were identified by gating on CD45 receptor (Panel A, Gate R16), and CLL cells were identified by gating on CD5 and CD19 receptor double-positive cells (Panel B, Gate R9). Expression of prognostic markers CD55 (Panel C), CD59 (Panel D), CD38 (Panel E), and ZAP-70 (Panel F) was then assessed on CLL cells by marker specific antibodies.

FIG. 2: (A) CLL cell counts at screen before starting treatment with FCR+L in patients who achieved (CR+PR, n=19) or did not achieve (SD+PD, n=11), a response. Data is presented in a vertical scatter plot with each dot representing a value from one patient in that group. Solid line represents the median value of each group. (B) CLL cell counts during treatment with up to 6 cycles of L+FCR in patients in the CR+PR (n=up to 20) versus SD+PD group (n=up to 11). Data is represented as median±interquartile range of values. * indicates statistical significant difference from screen values in each group (p<0.05; Wilcoxon Signed Rank test) and ** indicates significant difference between CR+PR and SD+PD group at each visit (p<0.05; Wilcoxon Rank Sum test).

FIG. 3: (A) Bar graph showing responses of patients to treatment with Lumiliximab in combination with fludarabine, cyclophosphamide and rituximab (L+FCR). Each bar represents treatment of an individual patient. The height of the bar indicates the percentage of CLL cells in a patient sample that express ZAP70. In all figures, CR, PR, SD and PD indicate complete response, partial response, stable disease and progressive disease respectively. (B) Levels (%) of ZAP-70⁺ CLL cells at screen before starting treatment with FCR+L in patients who achieved (CR+PR, n=12) or did not achieve (SD+PD, n=6) a response. Data is presented in a vertical scatter plot with each dot representing a value from one patient in that group. Solid line represents the median value of each group. The dotted line indicates value of 30% as a reference.

FIG. 4: (A) Bar graph showing caspase-3 activation in cells isolated from three patients before treatment and two days after treatment. (B) Western blot showing expression of ZAP70, phosphorylated ZAP70 and control (β-actin) proteins in samples isolated from the three patients represented in the bar graph.

FIG. 5: (A) Bar graph showing responses of patients to treatment with L+FCR. The height of each bar represents the percentage of CLL cells in a patient sample that express CD38. (B) Levels (%) of CD38⁺ CLL cells at screen before starting treatment with L+FCR in patients who achieved (CR+PR, n=19) or did not achieve (SD+PD, n=11) a response. Data is presented in a vertical scatter plot with each dot representing a value from one patient in that group. Solid line represents the median value of each group. The dotted line indicates a value of 30% as a reference. (C) Data is presented for the percentage of CD38⁺ CLL cells pre-treatment (screen), and at week 9, day 1 (W9D1). The results show the percentage in patients achieving or not achieving a response at screen. The results are show for a response at screen with <30% of CD38⁺ CLL cells at screen (CR+PR-Low) (n=7) or with >30% of CD38⁺ CLL cells at screen (CR+PR-High) (n=10), or not achieving a response with >30% of CD38⁺ CLL cells at screen (SD+PD-High) (n=5). Data is represented as median±range of values. The dotted line indicates a value of 30% as a reference. * indicates statistical significant difference from the screen value (p<0.05; Wilcoxon Signed Rank test)

FIG. 6: (A) Bar graph showing responses of patients to treatment with L+FCR. The height of each bar represents the amount of β2-microglobulin (in ng/ml) in patient samples. (B) Serum β2M at screen before starting treatment with FCR+L in patients who achieved (CR+PR, n=20) or did not achieve (SD+PD, n=11) a response. Data is presented in a vertical scatter plot with each dot representing a value from one patient in that group. Solid line represents the median value of each group. The dotted line indicates value of 2000 ng/ml as a reference. (C) Serum β2M levels pre-treatment (screen) and at week 13, day 1 (W13D1) in patients in the (CR+PR) n=17) versus SD+PD groups (n=5). Data is represented as median±range of values. The dotted line indicates a value of 2000 ng/ml as a reference. * indicates statistical significant difference from the screen value (p<0.05; Wilcoxon Signed Rank test)

FIG. 7: Bar graph showing responses of patients to treatment with L+FCR. The height of the bars represents the levels of soluble CD23 measured in patient samples obtained before treatment.

FIG. 8: Bar graphs showing responses of patients to treatment with L+FCR. In FIG. 8A, bar height indicates the percentage of CLL cells expressing CD23, and in FIG. 8B, bar height indicates the expression intensity of CD23 in CLL on cell cells.

FIG. 9: CD55 (A) and CD59 (C) expression (MFI) on CLL cells at screen before starting treatment with L+FCR in patients who achieved (CR+PR, n=19) or did not achieve (SD+PD, n=11) a response. Data is presented in a vertical scatter plot with each dot representing a value from one patient in that group. Solid line represents a median value of each group. CD55 (B) and CD59 (D) expression on CLL cells at screen and after 1 cycle in patients achieving (CR+PR, n=19) or not achieving SD+PD (n=11) a response. Data is represented as median±range of values. * indicates statistical significant difference from the screen value (p<0.05; Wilcoxon Signed Rank test).

FIG. 10. Lumiliximab induced downregulation of anti-apoptotic proteins including Bcl-2, Bcl-XL, Mcl-1, and XIAP in CLL cells. The effect of lumiliximab treatment on levels of anti-apopotic proteins were measured. FIG. 10A shows that the levels of Mcl-1, Bcl-XL, Bcl-2 were downregulated after lumilixumab treatment, whereas Bad, Bid, and β-actin control were not. FIG. 10B shows that the levels of XIAP were downregulated, whereas levels of cIAP-1, cIAP-2, survivin, and β-actin control were not.

### DETAILED DESCRIPTION OF THE INVENTION

### I. Definitions

It is to be noted that the term “a” or “an” entity refers to one or more of that entity; for example, “a CD23 antibody,” is understood to represent one or more CD23 antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to CD23 antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of CD23 antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Derivatives of CD23 antibodies and antibody polypeptides of the present invention, are polypeptides which have been altered so as to exhibit additional features not found on the native polypeptide. Examples include fusion proteins. Variant polypeptides may also be referred to herein as “polypeptide analogs.” As used herein a “derivative” of an CD23 antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an CD23 antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

The present invention is directed to certain CD23 antibodies, or antigen-binding fragments, variants, or derivatives thereof. Unless specifically referring to full-sized antibodies such as naturally-occurring antibodies, the term “CD23 antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2^{nd} ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an CD23 antibody or antigen-binding fragment, variant, or derivative thereof of the present invention are according to the Kabat numbering system.

In camelid species, the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library, and anti-idiotypic (anti-Id) antibodies (including, e.g., anti-Id antibodies to CD23 antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al. A human antibody is still “human” even if amino acid substitutions are made in the antibody.

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

In certain CD23 antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a VL or CL domain.

CD23 antibodies, or antigen-binding fragments, variants, or derivatives thereof disclosed herein may be described or specified in terms of the epitope(s) or portion(s) of an antigen, e.g., a target polypeptide (CD23) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by CD23 antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of CD23.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹. An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

CD23 antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

CD23 antibodies or antigen-binding fragments, variants or derivatives thereof of the invention may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

CD23 antibodies or antigen-binding fragments, variants or derivatives thereof of the invention may be “multispecific,” e.g., bispecific, trispecific or of greater multispecificity, meaning that it recognizes and binds to two or more different epitopes present on one or more different antigens (e.g., proteins) at the same time. Thus, whether an CD23 antibody is “monospecific” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an CD23 antibody, binding polypeptide or antibody. Each binding domain specifically binds one epitope. When an CD23 antibody, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments, the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., CD23 antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a binding molecule” and “an animal in need of treatment” includes subjects, such as mammalian subjects, that would benefit from administration of a binding molecule used, e.g., for detection of an antigen recognized by a binding molecule (e.g., for a diagnostic procedure) and/or from treatment, i.e., palliation or prevention of a disease such as cancer, with a binding molecule which specifically binds a given target protein. As described in more detail herein, the binding molecule can be used in unconjugated form or can be conjugated, e.g., to a drug, prodrug, or an isotope.

By “hyperproliferative disease or disorder” is meant all neoplastic cell growth and proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and “cancer.” In certain embodiments of the present invention, the hyperproliferative disease or disorder, e.g., the precancerous lesion, abnormal cell growth, benign tumor, malignant tumor, or “cancer” comprises cells which express, over-express, or abnormally express CD23.

As used herein, the term “malignancy” refers to a non-benign tumor or a cancer. As used herein, the term “cancer” connotes a type of hyperproliferative disease which includes a malignancy characterized by deregulated or uncontrolled cell growth. Examples of cancer include, but are not limited to, carcinoma, lymphoma, blastoma, sarcoma, and leukemia or lymphoid malignancies.

The method of the present invention may be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W.B. Saunders Co., Philadelphia, pp. 68-79 (1976). Such conditions in which cells begin to express, over-express, or abnormally express CD23, are particularly treatable by the methods of the present invention.

Hyperplasia is a form of controlled cell proliferation, involving an increase in cell number in a tissue or organ, without significant alteration in structure or function. Metaplasia is a form of controlled cell growth in which one type of adult or fully differentiated cell substitutes for another type of adult cell. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation.

### II. CD23 Antibodies

A “CD23 antibody” as used herein is an antibody that specifically binds to CD23. A CD23 antibody of the present invention can bind to transmembrane CD23 and/or to soluble CD23. In one embodiment, the CD23 antibody binds to human CD23.

In one embodiment of the invention, the CD23 antibody is lumiliximab (IDEC Pharmaceuticals, San Diego, Calif.). Lumiliximab is a is a chimeric macaque and human monoclonal CD23 antibody (also referred to herein as IDEC-152 or p5E8) against the CD23 antigen that has been developed for various indications (Nakumura and Kloetzer et al. 2:131 (2000)). Monoclonal antibody p5E8 originated from 5F8, a primate anti-human CD23 antibody secreting hybridoma from cynomolgus macaques and was molecularly cloned and expressed as a 150 kDa IgG monomer in CHO cells using proprietary vector technology. Monoclonal p5E8 maintains the 5E8 primate variable region coupled to the human γ1 heavy chain and human k light chain constant regions. It also retains C1q binding. The sequence and derivation of IDEC-152 and other potential antagonists are disclosed in U.S. Pat. No. 6,011,138 which is incorporated in its entirety herein by reference.

Other examples of antibodies that bind CD23 include the murine antibodies specific to CD23 such as MHM6 and the Primatized® antibodies specific to human CD23, including 5E8, 6G5, 2C8, B3B1 and 3G12, reported by Reff et al. in U.S. Pat. No. 6,011,138, antibodies and antibody fragments reported by Rector et al. J. Immunol. 55:481-488 (1985); Flores-Rumeo et al. Science 241:1038-1046 (1993); Sherr et al. J. Immunol., 142:481-489 (1989); and Pene et al., PNAS 85:6820-6824 (1988). Such antibodies are reportedly useful for treatment of allergy, autoimmune diseases, and inflammatory diseases. While existing CD23 antibodies may be used in the present invention, new antibodies may also be developed that are compatible with the disclosed methods using art recognized protocols.

### Poor Prognostic Markers and Methods of Assesing

According to the present invention, a “poor prognostic marker” is any protein or RNA whose expression levels can be correlated with rapid disease progression and/or short survival times. A mammal that “over expresses a poor prognostic marker” can have increased levels of the prognostic RNA or protein, or can have an increased number or percentage of cells that express the prognostic RNA or protein.

In one embodiment of the present invention, the poor prognostic marker is ZAP70 (zeta-chain (TCR) associated protein kinase 70 kDa). ZAP70 is a protein tyrosine kinase that is expressed T-cells and natural killer cells. It is believed to play a role in T-cell development and lymphocyte activation. It has been shown that ZAP70 mRNA and protein levels are higher in CLL patients than in healthy controls and also that ZAP70⁺ patients are characterized by unfavorable clinical results and shorter survival times (Durig et al. Leukemia 17:2426-34 (2003)). In particular, ZAP70 expression on greater than 30% of CLL cells in a patient is associated with a poor prognosis.

In other embodiment of the invention, the poor prognostic marker is CD38 (coiled-coil domain-containing protein 38). CD38 is a glycoprotein found on the surface of many immune cells, and its expression has also been identified as a CLL risk factor that can be used to identify patients with a poor prognosis (Hamblin et al. Blood 99:1023-1029 (2002)). In particular, CD38 expression on greater than 30% of CLL cells is associated with a poor prognosis.

In another embodiment of the invention, the poor prognostic marker is β2 microglobulin. β2 microglobulin is the beta-chain of major histocompatibility complex class I molecules. The normal range of β2 microglobulin levels is about 500-2000 ng/ml, and in CLL and immunocytoma (IC) patients, levels have been found to predict progression free survival (Hallek et al. Leuk. Lymphoma 22:439-47 (1996)). Levels of less than 3500 ng/ml in CLL patients are correlated with progression free survival (PFS).

In another embodiment of the invention, the poor prognostic marker is soluble CD23. Soluble CD23 is a cytokine that is thought to increase IL-4 induced IgE production from B cells. In healthy populations, average soluble CD23 levels are approximately 2.5 ng/ml, ranging from 1.5 to 5 ng/ml. However, it has been reported that soluble CD23 levels are higher in CLL patients than in healthy populations and that patients with high levels of soluble CD23 have a significantly worse prognosis than patients with lower levels (Sarfati et al. Blood 88:4259-4264 (1996)).

Methods for determining whether a mammal over expresses a poor prognostic marker are also contemplated by the present invention. Determining whether a mammal over expresses a poor prognostic marker may be done using methods known in the art. For example, techniques used to determine levels of protein or RNA expression can be used. In addition, techniques used to determine numbers or percentages of cells expressing a poor prognostic marker can be used.

Some embodiments of the invention involve a method of determining whether a mammal over expresses a poor prognostic marker by (a) obtaining a biological sample from the mammal, and (b) measuring the level of poor prognostic marker in the biological sample. In another embodiment the method of determining whether a mammal over expresses a poor prognostic marker comprises (a) obtaining a biological sample from the mammal, (b) measuring the level of poor prognostic marker in the biological sample and (c) comparing the level of poor prognostic marker to a reference sample.

Biological samples include, but are not limited to, blood, urine and CSF. Methods by which biological fluid samples may be obtained include, but are not limited to, tissue biopsy, venipuncture, urine collection and spinal tap. In one embodiment, the biological fluid sample is blood, serum or plasma.

The biological sample is obtained from a mammal. In some embodiments, the mammal is a vertebrate. Vertebrates include but are not limited to humans, mice, rats, sheep, goats, pigs, cattle, horses, reptiles, fishes, amphibians, and in eggs of birds, reptiles and fish. In one embodiment, the vertebrate is a human.

The level of poor prognostic marker can be measured by a number of assays. Any method known in the art for detecting proteins or RNA can be used. Such methods include, but are not limited to Coomassie Blue staining, immunodiffusion, immunoelectrophoresis, immunochemical methods, binder-ligand assays, immunohistochemical techniques, agglutination, complement assays, western blot analysis, northern blot analysis, and RT-PCR. (Basic and Clinical Immunology, 217-262, Sites and Terr, eds., Appleton & Lange, Norwalk, Conn., (1991), which is incorporated by reference).

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays, Western blot analysis and the like.

One embodiment of the present invention includes measuring the level of a poor prognostic marker by a Western blot, ELISA or Coomassie Blue staining assay. The Western blot analysis allows for the identification of specific proteins in which proteins from the sample are first separated by electrophoresis, transferred to nitrocellulose, and then immunoreacted with a protein-specific antibody. The ELISA analysis is a sensitive immunoassay that uses an enzyme linked to an antibody or antigen as a marker for the detection of a specific protein, especially another antigen or antibody. ELISAs are often used as a diagnostic test to determine exposure to a particular infectious agent, by identifying antibodies present in a blood sample. In the present invention, the ELISA can be used to determine levels of poor prognostic factors present in a biological sample. Coomassie Blue staining is a method used to identify proteins after a sample has been submitted to protein separation via electrophoresis. Both the Western blot and the ELISA require the use of an antibody or antigen binding fragment for protein detection. In one embodiment of the present invention, the level of poor prognostic marker is detected by an antibody or antigen binding fragment. In another embodiment, the level of poor prognostic marker is detected by a monoclonal antibody.

In determining expression of a poor prognostic marker in a patient, the level of the poor prognostic marker in the biological sample can be compared to the level of poor prognostic marker in a reference sample. A suitable reference sample can include, but is not limited to, a biological sample from a healthy individual. In one embodiment, the reference sample is from an individual not afflicted with a CD23⁺ malignancy or a leukemia or from an individual not afflicted with CLL.

Furthermore, poor prognostic markers in biological samples of individuals afflicted with a CD23⁺ malignancy or leukemia can vary in total level (i.e. total amount of protein or RNA) or in percentage of cells expressing the poor prognostic marker. In one embodiment of the present invention, the level of poor prognostic marker is elevated compared to the reference sample. In another embodiment, the level of poor prognostic marker is 1 fold greater than the reference sample. In another embodiment, the level of poor prognostic marker is 2 fold greater than the reference sample. In yet another embodiment, the level of poor prognostic marker is 3 fold greater than the reference sample. In another embodiment the poor prognostic marker is expressed on greater percentage of cells compared to the reference sample. In another embodiment, the percentage of cells expressing the poor prognostic marker is 1 fold greater than in the reference sample. In another embodiment, the percentage of cells expressing the poor prognostic marker is 2 fold greater than in the reference sample. In another embodiment, the percentage of cells expressing the poor prognostic marker is 3 fold greater than in the reference sample.

In another embodiment of the present invention, expression of a poor prognostic marker is defined by a finite quantity or RNA or protein expression. In another embodiment of the present invention, expression of a poor prognostic marker is defined by a finite percentage of cells expressing the poor prognostic marker. For example, in one embodiment of the present invention, the poor prognostic marker is expressed in 90%, 80%, 70%, 60%, 50%, 40%, 30% or 20% or greater of the cells in a biological sample obtained from a mammal. In another embodiment of the present invention, the poor prognostic marker is expressed in 90%, 80%, 70%, 60%, 50%, 40%, 30% or 20% or fewer of the cells in a biological sample obtained from a mammal.

### Methods of Treatment Using CD23 Antibodies

According to the present invention, CD23 antibodies can be used to treat any one of a number of CD23⁺ malignancies. As used herein, a CD23⁺ malignancy is any neoplasm wherein the neoplastic cells express or are associated with the CD23 antigen. Exemplary CD23⁺ neoplasms that may be treated in accordance with the present invention comprise relapsed Hodgkin's disease, resistant Hodgkin's disease high grade, low grade and intermediate grade non-Hodgkin's lymphomas, B cell chronic lymphocytic leukemia (B-CLL OR CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphomas, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkitt's and non-Burkitt's; follicular, predominantly large cell, follicular, predominantly small cleaved cell; and follicular, mixed small cleaved and large cell lymphomas. Still other neoplasms that may be treated with the compositions of the instant invention comprise T cell lymphomas, acute T cell leukemias and mastocytomas.

In another embodiment of the present invention CD23 antibodies can be used to treat leukemias, including B cell chronic lymphocytic leukemia (B-CLL OR CLL).

One embodiment of the present invention provides methods for treating a hyperproliferative disease or disorder, e.g., cancer, a malignancy, a tumor, or a metastasis thereof, in an animal suffering from such disease or predisposed to contract such disease, the method comprising, consisting essentially of, or consisting of administering to the animal an effective amount of an antibody or immunospecific fragment thereof, that binds to CD23 or a variant of CD23. Suitable antibodies include all antibodies and antigen-specific fragments thereof described herein. Examples include, but are not limited to, an isolated antibody or antigen-binding fragment thereof which specifically binds to the same CD23 epitope as a lumiliximab, an isolated antibody or antigen-binding fragment thereof which specifically binds to CD23, where the antibody or fragment thereof competitively inhibits lumiliximab from binding to CD23, or an isolated antibody or antigen-binding fragment thereof which specifically binds to CD23, where the antibody or fragment thereof comprises an antigen binding domain identical to that of lumiliximab.

In certain embodiments of the invention, the CD23 antibody, which can be lumiliximab, is used in conjunction with other pharmaceutically active agents. In one particular embodiment of the invention, lumilixmab is used in conjunction with fludarabine, cyclophosphamide and rituximab.

The terms “rituximab” or “RITUXAN®” herein refer to the genetically engineered chimeric murine/human monoclonal antibody directed against the CD20 antigen and designated “C2B8” in U.S. Pat. No. 5,736,B7, expressly incorporated herein by reference. The antibody is an IgG1 kappa immunoglobulin containing murine light and heavy chain variable region sequences and human constant region sequences. Rituximab has a binding affinity for the CD20 antigen of approximately 8.OnM and has shown efficacy in treating patients with CLL.

Fludarabine is a chemotherapy drug. It is a fluorinated nucleotide analog of the antiviral agent vidarabine. In vivo, a fludarabine metabolite appears to act by inhibiting DNA polymerase alpha, ribonucleotide reductase and DNA primase, and therefore inhibiting DNA synthesis. It is sold under the trade name Fludara®, which is approved for the treatment of adult patients with CLL who have not responded to or whose disease has progressed during treatment with at least one standard alkylating-agent containing regimen. According to the invention, fludarabine can be administered via injection, including by intravenous administration or by any other suitable route. Fludarabine can be administered together with a CD23 antibody as a single pharmaceutical composition, or they can be administered separately. According to the invention, fludarabine and the CD23 antibody can be administered according to the same dosing regimen or according to different dosing regimens.

Cyclophosphamide is an alkylating agent. Metabolites of cyclophosphamide interfere with the growth of rapidly proliferating malignant cells through a mechanism that is thought to involve cross-linking of DNA in the malignant cells. It is sold under the trade name Cytoxan®, which has been approved for the treatment of a number of malignancies including lymphomas, multiple myeloma, leukemias including CLL etc. According to the invention, cyclophosphamide can be administered via injection, including by intravenous administration, via oral administration, or by any other suitable route. Cyclophosphamide can be administered together with a CD23 antibody as a single pharmaceutical composition, or they can be administered separately. According to the invention, cyclophosphamide and the CD23 antibody can be administered according to the same dosing regimen or according to different dosing regimens.

In some embodiments an antibody of the present invention which specifically binds to CD23 or a variant thereof inhibits IgE from binding to CD23. In a further embodiment, an antibody of the present invention which specifically binds to CD23 or a variant thereof expressed on a cell, in particular a tumor cell or tumor associated macrophage, inhibits activation of downstream signal transduction molecules involved in cell proliferation or inhibition of apoptosis. In still a further embodiment, an antibody of the present invention which specifically binds to CD23 or a variant thereof expressed on a cell, in particular a tumor cell or tumor associated macrophage, inhibits the interaction of CD23 with IgE.

An antibody of the present invention which specifically binds to CD23 or a variant thereof, to be used in treatment methods disclosed herein can be prepared and used as a therapeutic agent that stops, reduces, prevents, or inhibits cellular activities involved in cellular hyperproliferation, e.g., cellular activities that induce the altered or abnormal pattern of vascularization that is often associated with hyperproliferative diseases or disorders.

Antibodies or immunospecific fragments thereof of the present invention include, but are not limited to monoclonal, chimeric or humanized antibodies, and fragments of antibodies that bind specifically to tumor-associated proteins such as CD23. The antibodies may be monovalent, bivalent, polyvalent, or bifunctional antibodies, and the antibody fragments include Fab F(ab′)₂, and Fv.

Therapeutic antibodies according to the invention can be used in unlabeled or unconjugated form, or can be coupled or linked to cytotoxic moieties such as radiolabels and biochemical cytotoxins to produce agents that exert therapeutic effects.

In certain embodiments, an antibody, or immunospecific fragment thereof of the invention includes an antigen binding domain. An antigen binding domain is formed by antibody variable regions that vary from one antibody to another. Naturally occurring antibodies comprise at least two antigen binding domains, i.e., they are at least bivalent. As used herein, the term “antigen binding domain” includes a site that specifically binds an epitope on an antigen (e.g., a cell surface or soluble antigen). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention provides methods for treating various hyperproliferative disorders, e.g., by inhibiting malignant cell growth or proliferation or by inducing apoptosis in malignant cells, in a mammal, comprising, consisting essentially of, or consisting of administering to the mammal an effective amount of a antibody or antigen-binding fragment thereof which specifically or preferentially binds to CD23, e.g., human CD23.

The present invention is more specifically directed to a method of treating a hyperproliferative disease, in an animal, e.g., a mammal, e.g., a human, comprising, consisting essentially of, or consisting of administering to an animal in need thereof an effective amount of a an antibody or immunospecific fragment thereof, which specifically or preferentially binds to one or more epitopes of CD23.

More specifically, the present invention provides a method of treating cancer in a human, comprising administering to a human in need of treatment a composition comprising an effective amount of an CD23-specific antibody or immunospecific fragment thereof, and a pharmaceutically acceptable carrier.

In certain embodiments, an antibody or fragment thereof binds specifically to at least one epitope of CD23 or fragment or variant, i.e., binds to such an epitope more readily than it would bind to an unrelated, or random epitope; binds preferentially to at least one epitope of CD23 or fragment or variant, i.e., binds to such an epitope more readily than it would bind to a related, similar, homologous, or analogous epitope; competitively inhibits binding of a reference antibody which itself binds specifically or preferentially to a certain epitope of CD23 or fragment or variant described above; or binds to at least one epitope of CD23 or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10¹⁰ M, about 10⁻¹ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M. In certain embodiments, antibodies and fragments thereof of the present invention cross-react with CD23 proteins of other species from which they were raised, e.g., an antibody or fragment thereof which specifically binds to human CD23 also binds to murine CD23. Other suitable antibodies or fragments thereof of the present invention include those that are highly species specific.

In specific embodiments, antibodies or immunospecific fragments thereof disclosed herein bind CD23 polypeptides or fragments or variants thereof with an off rate (k(off)) of less than or equal to 5×10⁻² sec⁻¹, 10⁻² sec⁻¹, 5×10⁻³ sec⁻¹ or 10⁻³ sec⁻¹. Other antibodies or immunospecific fragments thereof disclosed herein bind CD23 polypeptides or fragments or variants thereof with an off rate (k(off)) of less than or equal to 5×10⁻⁴ sec⁻¹, 10⁻⁴ sec⁻¹, 5×10⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, antibodies or immunospecific fragments thereof disclosed herein bind CD23 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Other antibodies or immunospecific fragments thereof for use in the diagnostic and treatment methods disclosed herein bind CD23 polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

In some embodiments of the present invention, the CD23 antibody of the present invention may be used in conjunction with additional pharmaceutically acceptable compounds that are effective in treating, preventing or inhibiting malignancies. Pharmaceutically acceptable compounds used in conjunction with a CD23 antibody according to the invention may be administered together in a single pharmaceutical composition or may be administered separately. Pharmaceutically acceptable compounds used in conjunction with a CD23 antibody according to the invention may be administered at the same time or at different times, and may be administered via the same route of administration or via different routes of administration.

It will further be appreciated that the CD23 antibody of the instant invention may be used in conjunction or combination with any chemotherapeutic agent or agents (e.g. to provide a combined therapeutic regimen) that eliminates, reduces, inhibits or controls the growth of neoplastic cells in vivo. As used herein the terms “chemotherapeutic agent” or “chemotherapeutics” shall be held to mean any therapeutic compound that is administered to treat or prevent the growth of neoplastic cells in vivo. In particular, chemotherapeutic agents compatible with the present invention comprise both “traditional” chemotherapeutic agents such as small molecules and more recently developed biologics such as antibodies, cytokines, antisense molecules, etc. that are used to reduce or retard the growth of malignant cells. Particularly preferred chemotherapeutic agents that are compatible for use with the disclosed CD23 antagonists include commercially available antibodies directed to tumor associated antigens such as Rituxan®, Zevalin™, Herceptin®, Lymphocide®, Campath®, etc. In additional preferred embodiments, antineoplastic antibodies undergoing clinical trials may be used in combination with CD23 antagonists. For example, IDEC-114 and IDEC-131 (IDEC Pharmaceuticals, San Diego Calif.) directed to the B7 antigen and CD40L antigen respectively, may be used with the disclosed antagonists to treat selected neoplasms. In this regard Lym-1 (Peregrin Pharmaceuticals, Tustin Calif.) and Erbitux™ (Imclone Pharmaceuticals, Cambridge Mass.) are also compatible with the instant invention. Other biologic chemotherapeutic agents that are compatible include cytokines such as lymphokines, interleukins, tumor necrosis factors and growth factors. The CD23 antagonists may also be used in conjunction with immunosuppressive agents, prodrugs or cytotoxic agents for the treatment of selected malignancies.

Chemotherapeutic antibodies that are particularly useful in combination with CD23 antagonists include Y2B8 and C2B8 (Zevalin™ & Rituxan®) IDEC-114 and IDEC-131, (IDEC Pharmaceuticals Corp., San Diego), Lym 1 and Lym 2, LL2 (Immunomedics Corp., New Jersey), HER2 (Herceptin®, Genentech Inc., South San Francisco), B1 (Bexxar®, Coulter Pharm., San Francisco), MB1, BH3, B4, B72.3 (Cytogen Corp.), CC49 (National Cancer Institute) and 5E10 (University of Iowa). Rituxan is the first FDA-approved monoclonal antibody for treatment of human B-cell lymphoma (see U.S. Pat. Nos. 5,843,439; 5,776,456 and 5,736,137 each of which is incorporated herein by reference). Y2B8 is the murine parent of C2B8. Rituxan is a chimeric, anti-CD20 monoclonal antibody (MAb) which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83:435-445 (1994)). Those skilled in the art will appreciate that any antibody directed to common tumor associated or immunomodulatory antigens such as CD20, CD22, B7 or CD40L is compatible with the instant invention and may be used in combination with the disclosed antagonists.

More “traditional” chemotherapeutic agents useful in the instant invention include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime nitrogen mustards such as chiorambucil, chlomaphazine, cyclosphosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol: pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (Taxotere, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and antiandrogens such as flutamide, nilutamide, bicalutamide, leuprolide, and goserelin; and pharmaceutically acceptable salts, acids or derivatives of any of the above.

Compatible chemotherapeutic regimens of comprise combinations of drugs. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphoma, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with the CD23 antagonists as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For some patients salvage therapy is used. Salvage therapies employ drugs such as cytosine arabinoside, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, IID cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules. The amount of chemotherapeutic agent to be used in combination with the CD23 antagonists of the instant invention may vary by subject or may be administered according to what is known in the art. See for example, Bruce A Chabner et al, Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens. Examples of such agents include 2-amino-6-aryl-5-substituted pyrimidines (see U.S. Pat. No. 4,665,077, the disclosure of which is incorporated herein by reference), azathioprine; cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as glucocorticosteroids, e.g. prednisone, methylpredisolone, and dexamethasone; cytokine or cytokine receptor antagonists including anti-interfeCD23 antibodies, anti-tumor necrosis factor-.alpha. antibodies, anti-tumor necrosis factor-β antibodies, anti-interleukin-2 antibodies and anti-IL-2 receptor antibodies; anti-LFA-1 antibodies, including anti-CD1 1a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990), streptolanase; TGF-β; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al., Science, 251:430-432 (1991); WO 90/11294; laneway, Nature, 341:482 (1989); and WO 91/01133); and T cell receptor antibodies (EP 340,109) such as T10B9.

The term “cytotoxic agent” as used herein refers to a substance that inhibits or prevents the function of cells and/or causes destruction of cells. The term is intended to include radioactive isotopes, chemotherapeutic agents, and toxins such as small molecule toxins or enzymatically active toxins of bacterial, fungal, plant or animal origin, or fragments thereof.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interfeCD23-α, -β, and -γ; colony stimulating factors (CSFs) such as macrophage-CSF (M-CSF); granulocytemacrophage-CSF (GM-CSF); and granulocyte-CSF (G-CSF); interleukins (ILs) such as IL-1, IL-1a, IL-2, IL-g, IL-4, IL-5, IL-6, IL-7, IL-8, IL-9, IL11, IL-12, IL-15; a tumor necrosis factor such as TNF-α. or TNF-β; and other polypeptide factors including LIF and kit ligand (KL). As used herein, the term cytokine includes proteins from natural sources or from recombinant cell culture and biologically active equivalents of the native sequence cytokines.

In one embodiment of the invention, the CD23 antibody of the present invention is used in conjunction with fludarabine, cyclophosphamide and rituximab.

### Pharmaceutical Compositions and Methods of Administration

Methods of preparing and administering CD23-specific antibodies or immunospecific fragments thereof to a subject in need thereof are well known to or are readily determined by those skilled in the art. The route of administration of the binding molecule, e.g., binding polypeptide, e.g., CD23-specific antibody or immunospecific fragment thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, binding molecules, e.g., binding polypeptides, e.g., CD23-specific antibodies or immunospecific fragments thereof can be delivered directly to the site of the adverse cellular population thereby increasing the exposure of the diseased tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a binding molecule, e.g., a binding polypeptide, e.g., CD23-specific antibody or immunospecific fragment thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 (US-2002-0102208 A1), which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present invention, for treatment of hyperproliferative disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of hyperproliferative disorders with an antibody or fragment thereof, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

CD23-specific antibodies or immunospecific fragments thereof disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, binding molecules can be administered as a sustained release formulation, in which case less frequent administration is required. Dosage and frequency vary depending on the half-life of the antibody in the patient. The half-life of a binding molecule can also be prolonged via fusion to a stable polypeptide or moiety, e.g., albumin or PEG. In general, humanized antibodies show the longest half-life, followed by chimeric antibodies and nonhuman antibodies. In one embodiment, the binding molecules of the invention can be administered in unconjugated form, In another embodiment, the binding molecules, e.g., binding polypeptides, e.g., CD23-specific antibodies or immunospecific fragments thereof for use in the methods disclosed herein can be administered multiple times in conjugated form. In still another embodiment, the binding molecules of the invention can be administered in unconjugated form, then in conjugated form, or vise versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an CD23-specific antibody or immunospecific fragment thereof (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. In some methods, agents are injected directly into a particular tissue where CD23-expressing cells have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device, such as a Medipad™ device.

CD23 antibodies or fragments thereof of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled binding polypeptides range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of the instant invention include, but are not limited to, ¹²³I, ¹²⁵I, ³²P, ⁵⁷Co, ⁶⁴Cu, ⁶⁷CU, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au ²²⁵Ac, ²¹¹At, and ²¹³Bi respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy (Peirersz et al. Immunol. Cell Biol. 65: 111-125 (1987)). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

Whether or not CD23-specific antibodies or immunospecific fragments thereof disclosed herein are used in a conjugated or unconjugated form, it will be appreciated that a major advantage of the present invention is the ability to use these molecules in myelosuppressed patients, especially those who are undergoing, or have undergone, adjunct therapies such as radiotherapy or chemotherapy. That is, the beneficial delivery profile (i.e. relatively short serum dwell time, high binding affinity and enhanced localization) of the molecules makes them particularly useful for treating patients that have reduced red marrow reserves and are sensitive to myelotoxicity. In this regard, the unique delivery profile of the molecules make them very effective for the administration of radiolabeled conjugates to myelosuppressed cancer patients. As such, the CD23-specific antibodies or immunospecific fragments thereof disclosed herein are useful in a conjugated or unconjugated form in patients that have previously undergone adjunct therapies such as external beam radiation or chemotherapy. In other preferred embodiments, binding molecules, e.g., binding polypeptides, e.g., CD23-specific antibodies or immunospecific fragments thereof (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies or other binding molecules and one or more chemotherapeutic agents. Particularly preferred embodiments of this aspect of the invention will comprise the administration of a radiolabeled binding polypeptide.

While CD23-specific antibodies or immunospecific fragments thereof may be administered as described immediately above, it must be emphasized that in other embodiments conjugated and unconjugated binding molecules may be administered to otherwise healthy patients as a first line therapeutic agent. In such embodiments binding molecules may be administered to patients having normal or average red marrow reserves and/or to patients that have not, and are not, undergoing adjunct therapies such as external beam radiation or chemotherapy.

However, as discussed above, selected embodiments of the invention comprise the administration of CD23-specific antibodies or immunospecific fragments thereof to myelosuppressed patients or in combination or conjunction with one or more adjunct therapies such as radiotherapy or chemotherapy (i.e. a combined therapeutic regimen). As used herein, the administration of CD23-specific antibodies or immunospecific fragments thereof in conjunction or combination with an adjunct therapy means the sequential, simultaneous, coextensive, concurrent, concomitant or contemporaneous administration or application of the therapy and the disclosed binding molecules. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates described herein. Conversely, cytotoxin-conjugated binding molecules could be administered intravenously followed by tumor localized external beam radiation. In yet other embodiments, binding molecules may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of a binding molecule (with or without cytotoxin) and the chemotherapeutic agent may be administered in any order and within any time frame that provides a therapeutic benefit to the patient. That is, the chemotherapeutic agent and CD23-specific antibody or immunospecific fragment thereof, may be administered in any order or concurrently. In selected embodiments CD23-specific antibodies or immunospecific fragments thereof of the present invention will be administered to patients that have previously undergone chemotherapy. In yet other embodiments, CD23-specific antibodies or immunospecific fragments thereof of the present invention will be administered substantially simultaneously or concurrently with the chemotherapeutic treatment. For example, the patient may be given the binding molecule while undergoing a course of chemotherapy. In preferred embodiments the binding molecule will be administered within 1 year of any chemotherapeutic agent or treatment. In other preferred embodiments the polypeptide will be administered within 10, 8, 6, 4, or 2 months of any chemotherapeutic agent or treatment. In still other preferred embodiments the binding molecule will be administered within 4, 3, 2 or 1 week of any chemotherapeutic agent or treatment. In yet other embodiments the binding molecule will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

Moreover, in accordance with the present invention a myelosuppressed patient shall be held to mean any patient exhibiting lowered blood counts. Those skilled in the art will appreciate that there are several blood count parameters conventionally used as clinical indicators of myelosuppression and one can easily measure the extent to which myelosuppression is occurring in a patient. Examples of art accepted myelosuppression measurements are the Absolute Neutrophil Count (ANC) or platelet count. Such myelosuppression or partial myeloablation may be a result of various biochemical disorders or diseases or, more likely, as the result of prior chemotherapy or radiotherapy. In this respect, those skilled in the art will appreciate that patients who have undergone traditional chemotherapy typically exhibit reduced red marrow reserves. As discussed above, such subjects often cannot be treated using optimal levels of cytotoxin (i.e. radionuclides) due to unacceptable side effects such as anemia or immunosuppression that result in increased mortality or morbidity.

More specifically conjugated or unconjugated CD23-specific antibodies or immunospecific fragments thereof of the present invention may be used to effectively treat patients having ANCs lower than about 2000/mm³ or platelet counts lower than about 150,000/mm³. More preferably CD23-specific antibodies or immunospecific fragments thereof of the present invention may be used to treat patients having ANCs of less than about 1500/mm³, less than about 1000/mm³ or even more preferably less than about 500/mm³. Similarly, CD23-specific antibodies or immunospecific fragments thereof of the present invention may be used to treat patients having a platelet count of less than about 75,000/mm³, less than about 50,000/mm³ or even less than about 10,000/mm³. In a more general sense, those skilled in the art will easily be able to determine when a patient is myelosuppressed using government implemented guidelines and procedures.

As indicated above, many myelosuppressed patients have undergone courses of treatment including chemotherapy, implant radiotherapy or external beam radiotherapy. In the case of the latter, an external radiation source is for local irradiation of a malignancy. For radiotherapy implantation methods, radioactive reagents are surgically located within the malignancy, thereby selectively irradiating the site of the disease. In any event, CD23-specific antibodies or immunospecific fragments thereof of the present invention may be used to treat disorders in patients exhibiting myelosuppression regardless of the cause.

In this regard it will further be appreciated that CD23-specific antibodies or immunospecific fragments thereof of the present invention may be used in conjunction or combination with any chemotherapeutic agent or agents (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasias in such patients. In other embodiments, radiolabeled immunoconjugates disclosed herein may be effectively used with radiosensitizers that increase the susceptibility of the neoplastic cells to radionuclides. For example, radiosensitizing compounds may be administered after the radiolabeled binding molecule has been largely cleared from the bloodstream but still remains at therapeutically effective levels at the site of the tumor or tumors.

With respect to these aspects of the invention, exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in Harrison's Principles of Internal Medicine 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more CD23-specific antibodies or immunospecific fragments thereof of the present invention.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade malignancies, who fail to achieve remission or relapse, salvage therapy is used. Salvage therapies employ drugs such as cytosine arabinoside, cisplatin, carboplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the CD23-specific antibodies or immunospecific fragments thereof of the present invention may vary by subject or may be administered according to what is known in the art. See, for example, Bruce A Chabner et al., Antineoplastic Agents, in Goodman & Gilman's The Pharmacological Basis of Therapeutics 1233-1287 (Joel G. Hardman et al., eds., 9^{th} ed. (1996)).

In another embodiment, an CD23-specific antibody or immunospecific fragment thereof of the present invention is administered in conjunction with a biologic. Biologics useful in the treatment of cancers are known in the art and a binding molecule of the invention may be administered, for example, in conjunction with such known biologics.

For use in treatment of Non-Hodgkin's Lymphomas currently approved therapies include: Bexxar® (tositumomab and iodine I-131 tositumomab, GlaxoSmithKline, Research Triangle Park, N.C.; a multi-step treatment involving a mouse monoclonal antibody (tositumomab) linked to a radioactive molecule (iodine I-131)); Intron® A (interfeCD23 alfa-2b, Schering Corporation, Kenilworth, N.J.; a type of interfeCD23 approved for the treatment of follicular non-Hodgkin's lymphoma in conjunction with anthracycline-containing combination chemotherapy (e.g., cyclophosphamide, doxorubicin, vincristine, and prednisone [CHOP])); Rituxan® (rituximab, Genentech Inc., South San Francisco, Calif., and Biogen Idec, Cambridge, Mass.; a monoclonal antibody approved for the treatment of non-Hodgkin's lymphoma; Ontak® (denileukin diftitox, Ligand Pharmaceuticals Inc., San Diego, Calif.; a fusion protein consisting of a fragment of diphtheria toxin genetically fused to interleukin-2); and Zevalin® (ibritumomab tiuxetan, Biogen Idec; a radiolabeled monoclonal antibody approved by the FDA for the treatment of B-cell non-Hodgkin's lymphomas).

For treatment of Leukemia, exemplary biologics which may be used in combination with the binding molecules of the invention include Gleevec®; Campath®-1H (alemtuzumab, Berlex Laboratories, Richmond, Calif.; a type of monoclonal antibody used in the treatment of chronic Lymphocytic leukemia). In addition, Genasense (oblimersen, Genta Corporation, Berkley Heights, N.J.; a BCL-2 antisense therapy under development to treat leukemia may be used (e.g., alone or in combination with one or more chemotherapy drugs, such as fludarabine and cyclophosphamide) may be administered with the claimed binding molecules.

For the treatment of multiple myeloma, exemplary biologics include Velcade® Velcade (bortezomib, Millennium Pharmaceuticals, Cambridge Mass.; a proteasome inhibitor). Additional biologics include Thalidomid® (thalidomide, Clegene Corporation, Warren, N.J.; an immunomodulatory agent and appears to have multiple actions, including the ability to inhibit the growth and survival of myeloma cells and anti-angiogenesis).

Other exemplary biologics include the MOAB IMC-C225, developed by InClone Systems, Inc., New York, N.Y.

As previously discussed, CD23-specific antibodies or immunospecific fragments thereof of the present invention, or recombinants thereof may be administered in a pharmaceutically effective amount for the in vivo treatment of mammalian hyperproliferative disorders. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of CD23-specific antibodies or immunospecific fragments thereof of the present invention, or recombinant thereof, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the binding molecule will be preferably be capable of interacting with selected immunoreactive antigens on neoplastic or immunoreactive cells, or on non neoplastic cells, e.g., vascular cells associated with neoplastic cells. and provide for an increase in the death of those cells. Of course, the pharmaceutical compositions of the present invention may be administered in single or multiple doses to provide for a pharmaceutically effective amount of the binding molecule.

In keeping with the scope of the present disclosure, CD23-specific antibodies or immunospecific fragments thereof of the present invention may be administered to a human or other animal in accordance with the aforementioned methods of treatment in an amount sufficient to produce a therapeutic or prophylactic effect. The CD23-specific antibodies or immunospecific fragments thereof of the present invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding molecules according to the present invention may prove to be particularly effective.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### Material and Methods

### Patient Demographics and Treatment Assignment

Thirty-one patients with symptomatic, previously treated CLL were enrolled. Patients were assigned sequentially to cohorts 1, 2, or a phase II expansion cohort that expanded the cohort 2 experience. In cycle 1, patients received rituximab 50 mg/m² IV on day 1 and 325 mg/m² IV on day 3; lumiliximab 50 mg/m² IV on day 2 and 325 mg/m²⁴ (cohort 1, n=3) or 450 mg/m² (cohort 2 or phase II expansion, n=28) IV on day 4; fludarabine 25 mg/m² IV on days 2, 3, and 4; and cyclophosphamide 250 mg/m² IV days 2, 3, and 4. In cycles 2-6, patients were administered rituximab 500 mg/m² IV on day 1; lumiliximab 375 mg/m² (cohort 1) or 500 mg/m² (cohort 2 or phase II expansion) IV on day 1; fludarabine 25 mg/m² IV on days 1, 2, and 3; and cyclophosphamide 250 mg/m² IV on days 1, 2, and 3. All patients received allopurinol for the first 10 days of therapy, Granisetron hydrochloride (or equivalent) on each day of therapy, and acetaminophen and diphenhydramine hydrochloride before each dose of antibody therapy. Additional supportive care was administered at the discretion of the treating physician.

### β2M

β2M was measured at PRL using the IMMULITE β2M In-Vitro Diagnostic Test Kit and the IMMULITE Automated Analyzer. Briefly, polystyrene beads coated with anti-β2M antibody were incubated (dilution-1:41) with an alkaline phosphatase-conjugated monoclonal antibody for 30 minutes at 37° C. into the Test Unit with intermittent agitation to allow formation of antibody sandwich complex with β2M in the sample. Unbound conjugate was then removed by a centrifugal wash, and a chemiluminescent substrate was added to the Test Unit. The light emitted by the bound complex, which is proportional to the concentration of β2M in the sample, was measured and recorded. The concentration of β2M in samples was extrapolated from a standard curve generated with each assay.

### IgV_{H} Mutation Status

Peripheral blood or bone marrow specimens were submitted to Genoptix (San Diego, Calif.) in BD Vacutainer tube containing EDTA as an anticoagulant (Becton-Dickinson, San Jose, Calif.). From the peripheral blood specimens, peripheral blood mononuclear cells (PBMC) were prepared by density gradient centrifugation and PBMC cell pellets were snap-frozen at −80° C. Genomic DNA was prepared from snap-frozen PBMC pellet using QIAamp DNA Blood Mini Kit (Qiagen, Hilden, Germany). IgV_{H} region was amplified using Hypermutation mix 1 containing 5′ VH leader primers (L) and 3′ JH primers according to InVivoScribe IGH somatic hypermutation manual (InVivoScribe Technologies, LLC, San Diego, Calif.). In samples where one discrete PCR band was observed by PAGE, the PCR reaction mix was purified using QIAquick gel extraction kit (Qiagen, Hilden, Germany) and sequenced using JH primer. Sequence alignment was performed using IMGT/V-QUEST database (imgt.cines.fr/IMGT_vquest/share/textes/index html) according to ERIC recommendation. Ghia et al., Leukemia 21:1-3 (2007). The percentage of deviation was calculated based on the ratio between the number of nucleotide differences, that is, mutations within the IgV_{H} region of the IgV_{H}-D-J rearrangement sequence, and the length in nucleotide of the most homologous germ-line IgV_{H} gene from the first (FR1-IMGT or Kabat codon 1) to the last codon (CDR3-IMGT codon 105/106/107 depending on exonuclease trimming) of the IgV_{H} gene. A change in the last codon was counted as a mutation only if occurring at the first nucleotide position. The homology was then calculated by 1% deviation. A 98% homology cut-off was used to determine whether the IgV_{H} gene is mutated (<98%) or unmutated (≧98%).

### Flow Cytometry: CD38, CD55, and CD59

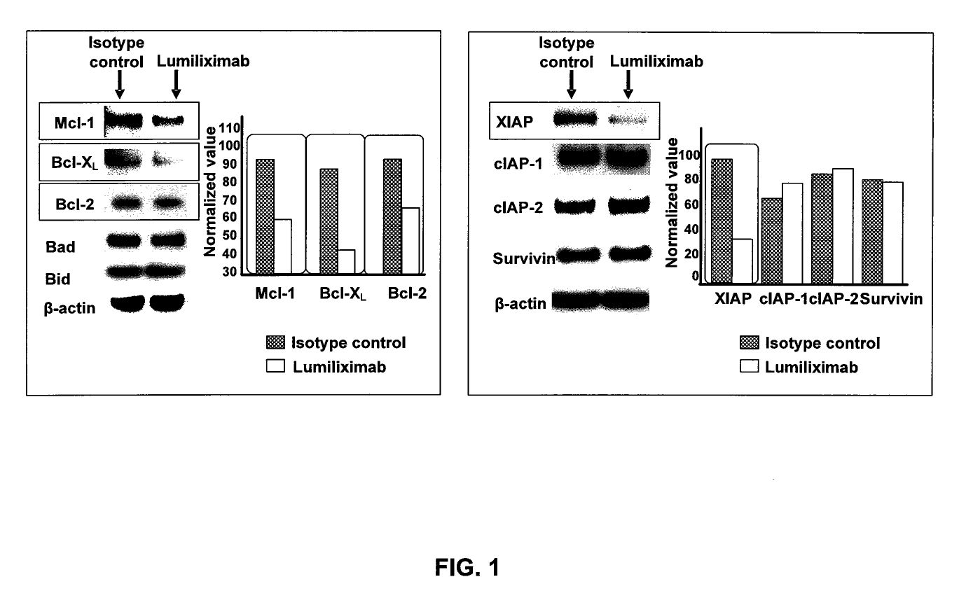

Whole blood was collected pre- and post-treatment into EDTA Vacutainer tubes and shipped within 24 hours of collection to Biogen Idec in San Diego. Whole blood counts were determined using the Sysmex Automated Hematology machine, diluted to a concentration of 10×10⁶/ml and incubated with the antibody of interest for 20 minutes at room temperature in the dark. The antibodies used include CD45-PerCP (clone 2D1 BD Biosciences), CD5-FITC (clone L17F12 BD Bioscience,), CD19-APC (clone HIB19, BD Bioscience), CD38-PE (clone HB.7, BD Bioscience), CD55-PE (clone IA10, BD Bioscience), and CD59-PE (clone H19, BD Bioscience). Four-color flow cytometry (FACScalibur (Becton Dickinson)) analysis was used to assess the absolute counts (cells/mm³) of CD5+/CD19+ CLL cells and the frequency of CD38+ CLL cells. Mean fluorescence intensity (MFI) was utilized to measure the expression level of the CD55 and CD59 antigen on the surface of CLL cells. Lymphocytes were first gated on CD45 and side scatter (SSC), followed by identification of CLL cells by gating on the CD5+/CD19+ population (FIG. 1). The expression of antigens CD38, CD55, and CD59 was then assessed on the CLL gated population and data were reported as % of total CLL cells (CD38) or MFI (CD55 and CD59). A cut-off value of 30% was used to classify patients in high CD38 expression group.

### Flow Cytometry: ZAP-70

Whole blood (pre-treatment) was first stained with cell surface antibodies, as described above, then permeabilized (Fix & Perm Cell Permeabilizaiton Kit, Caltag Laboratories). The cells were then incubated with 1.5 μg of anti-ZAP-70 antibody (clone 1E7.2, eBiosciences, cat# 11-6695) per 500,000 cells for 20 minutes at room temperature in the dark, and fixed in 0.2% paraformaldehyde. Cells were then analyzed by flow cytometry by first gating on the CD3+CD56+ cells, as an internal control for ZAP-70 expression, then gated on the CD5+/CD19+ CLL cells. Determination of ZAP-70⁺ cells was made by gating on CD5+/CD19+ cells (FIG. 1) and the data were reported as % of total CLL cells (ZAP-70⁺). The samples were processed for ZAP-70 expression only if the analysis could be done within 24 hours of collection. A 30% cut-off value was used for the analysis of ZAP-70 expressing CLL cells.

### Data Analysis

Patients were assessed at weeks 13 and 25 for response, which was defined using the NCIWG 96 criteria for CLL and reported as either complete response (CR), partial response (PR), stable disease (SD), or progressive disease (PD). Twenty patients responded to treatment with either a CR(N=16) or a PR(N=4); whereas, 11 patients were categorized as either SD (N=2) or PD. Because of a small study population, patients were divided into 2 groups based on response: (1) who responded (CR+PR) and (2) who did not respond (SD+PD) to FCR+L. The prognostic markers in these groups were assessed at screening and at various time points during the study. For CD55 and CD59, an average of MFI at weeks 5 and 9 (after 1 cycle of treatment) was considered due to fewer patients evaluated at either visits. Because of a small sample size and non-normal distribution of data, median and range values are reported and non-parametric tests (Wilcoxon Rank Sum test and Wilcoxon Signed Rank test for continuous variables and Fisher's Exact test for categorical variables) are used to analyze significant difference between groups and within groups at various time points. A p value of <0.05 was considered to be statistically significant.

### EXAMPLES

### Example 1

### Tumor Burden in Patients Enrolled in 152-30 Study

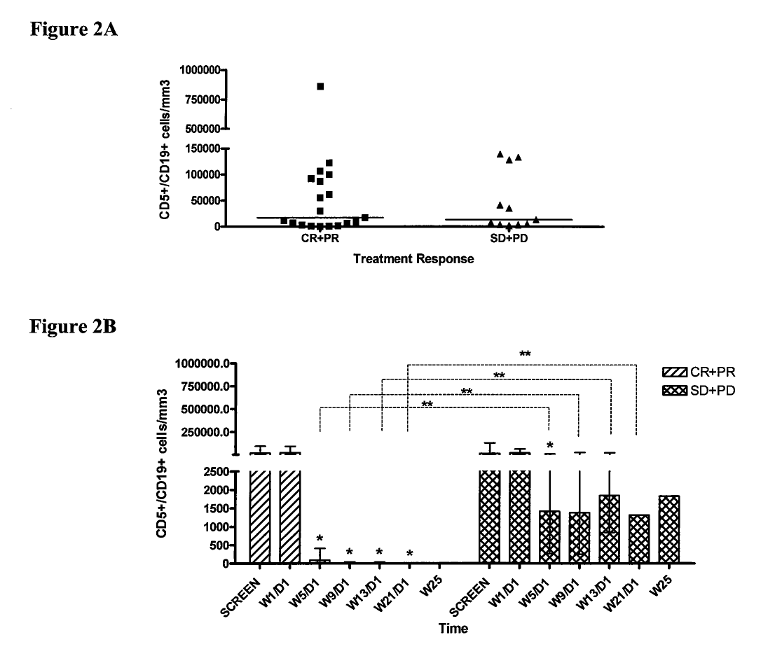

In order to determine if lumiliximab, in combination with fludarabine, cyclophosphamide and rituximab (L+FCR) is effective in treating patients with poor prognosis, CLL cells were isolated from patients pre-treatment. The pre-treatment tumor burden as assessed by the number of CD5+/CD19+ cells at screen in patients in the CR+PR group was not significantly different from those in the SD+PD group (p=0.8311; Wilcoxon Rank Sum test), suggesting that high tumor load did not have a negative impact on treatment outcome (FIG. 2A). In addition, the number of CD5+/CD19+ cells progressively declined over the course of follow-up, starting from week 5 after treatment initiation, in both the CR+PR and SD+PD group (FIG. 2B). However, at weeks 5, 9, 13 and 21 after the initiation of treatment, the number of CD5+/CD19+ cells were significantly lower in the CR+PR group compared to SD+PD group as predicted (p<0.05 for each week; Wilcoxon Signed Rank test).

### Example 2

### Lumiliximab Combination Treatment is Effective in Patients with High ZAP70 Expression

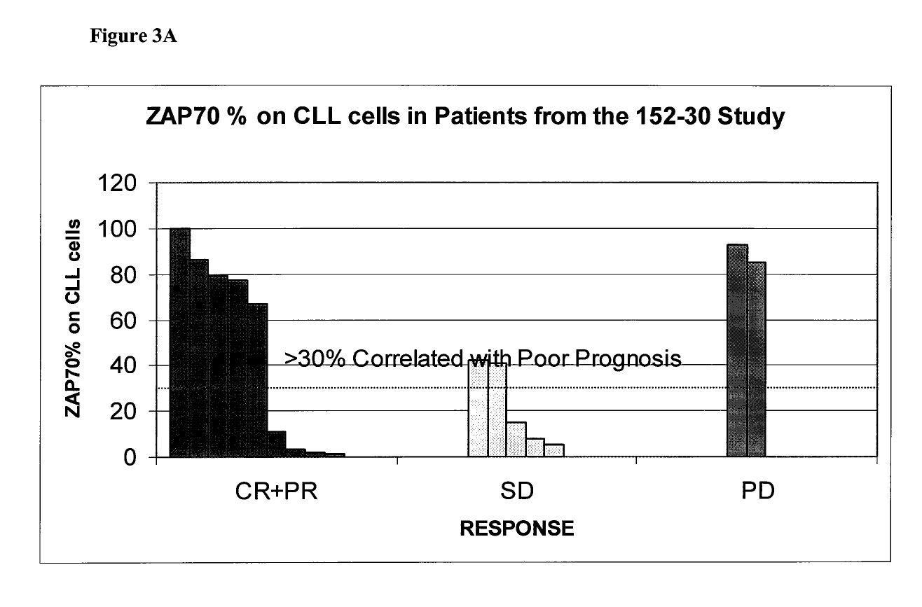

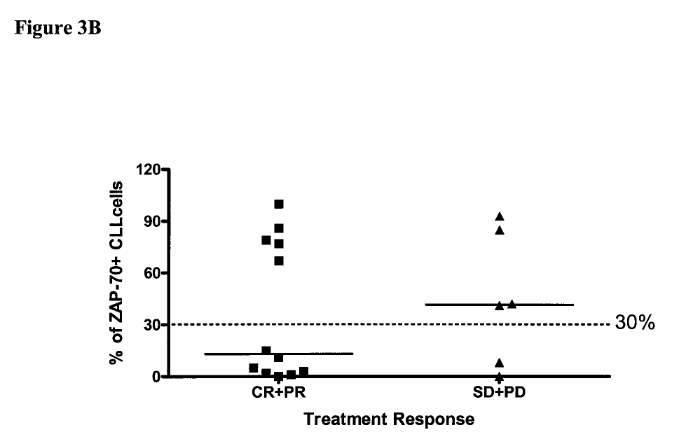

The CLL samples were used to determine the presence or absence of poor prognostic markers. For example, it has previously been shown that ZAP70 expression on at least 30% of CLL cells is associated with a poor prognosis. Therefore, the percentage of ZAP70⁺ CLL cells in the patient samples was measured using flow cytometry. Nine of the sixteen patients tested had more than 30% ZAP⁺ CLL cells. Patients were treated with L+FCR and their response was assessed. Patients were classified as complete responders (CR), partial responders (PR), stable disease (SD) or progressive disease (PD). The results are shown in FIG. 3A. Five of the nine patients with greater than 30% ZAP⁺ CLL were complete or partial responders. Interestingly, the five patients that responded well to treatment had greater than 60% ZAP70⁺ CLL cells.

After the inclusion of additional patients in the study, eighteen patients had ZAP-70 results reported at screening. The percentage of ZAP-70⁺ cells at screen was not significantly different (p=0.7124; Wilcoxon Rank Sum test) in the CR+PR group compared to the SD+PD group (FIG. 3B). When the same data was categorized using the cut-off value, 5 out of 12 patients (42%) had over 30% of ZAP-70⁺ cells in CR+PR group compared to 67% (4 out of 6) in the SD+PD group; this difference was not found to be statistically significant (p=0.6199; Fisher's Exact test). These results indicate that L+FCR treatment is effective in treating patients with high ZAP70 expression.

### Example 3

### Lumiliximab Induces Apoptosis in ZAP70⁺ Cells

It is thought that lumiximab can induce apoptosis by activating caspases.

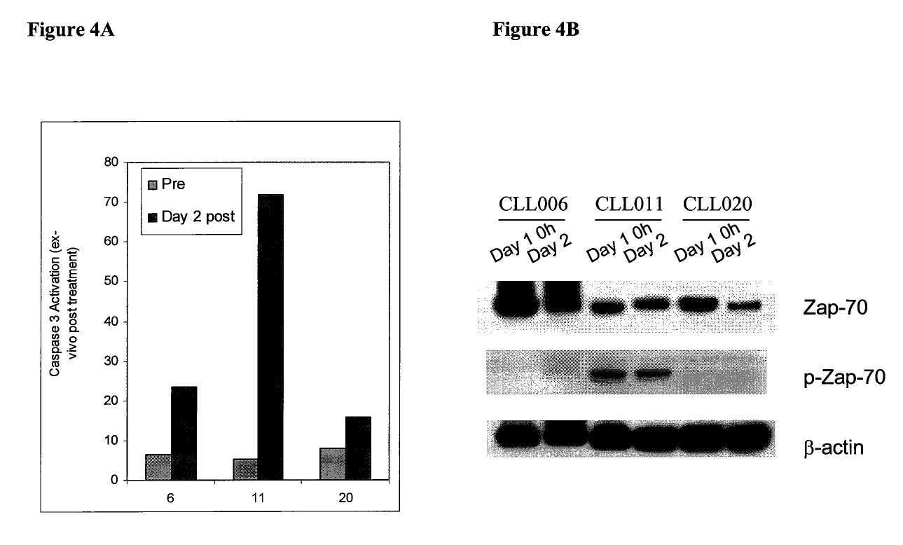

Therefore, in order to determine if lumiliximab can induce apoptosis in ZAP70⁺ cells, activation of caspase-3 in ZAP70⁺ cells was measured before and after treatment with lumiliximab. The results are shown in FIG. 4A. Expression of ZAP70 was assessed by western blots using antibodies against ZAP70, phosphorylated ZAP70 and β-actin as a control. ZAP70 expression was seen in all three patient samples tested, but levels of protein expression and phosphorylation varied between patients (see FIG. 4B). However, caspase-3 activity was higher two days after treatment than before treatment in samples from all three patients. These results indicate that lumiliximab can induce apoptosis in cells expressing ZAP70 and/or phosphorylated ZAP70.

### Example 4

### Lumiliximab Combination Treatment is Effective in Patients with High CD38 Expression

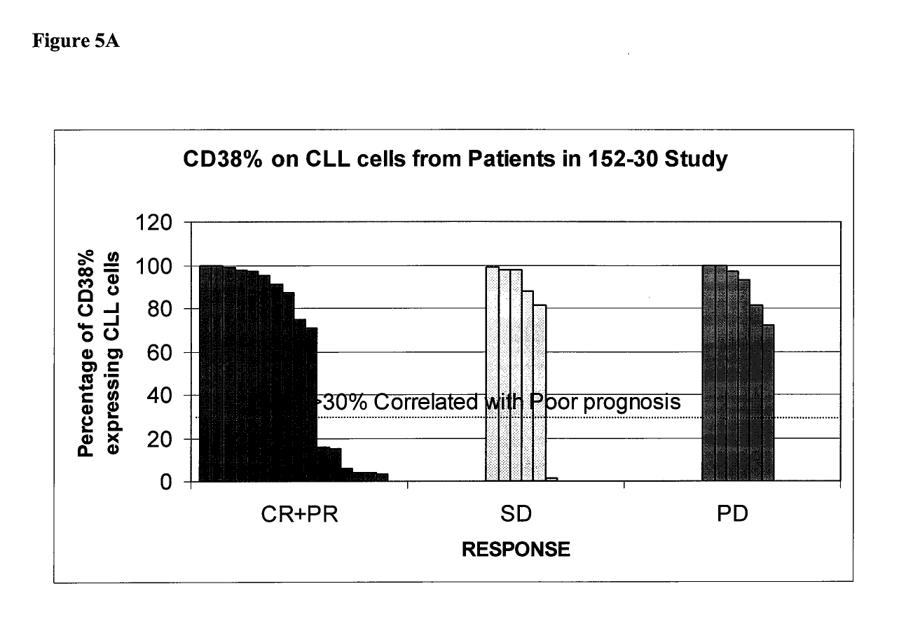

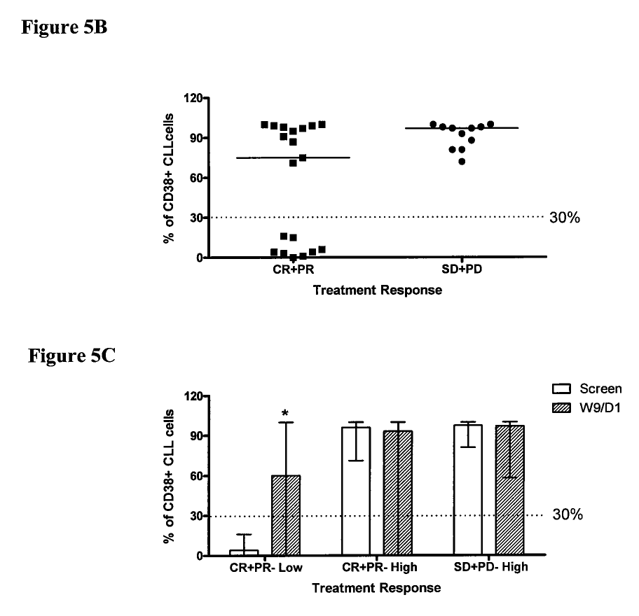

In order to determine if L+FCR treatment is also effective in treating patients with other poor prognostic markers, the efficacy of treatment was compared to levels of CD38 expression. Patients were treated with L+FCR as described in Example 1. Expression of CD38 on at least 30% of CLL cells has previously been associated with a poor prognosis. However, even patients with greater than 60% CD38⁺ cells responded completely or partially to L+FCR treatment. See FIG. 5A.

After the inclusion of additional patients in the study, out of 30 patients, who had adequate sample for CD38 analysis at screen, 8 patients (27%) had fewer than 30% CD38⁺ cells. All of these patients belonged to CR+PR group, which was further divided into two sub groups, namely (1)<30% CD38⁺ cells (CR+PR-low, n=8 (42%)) and (2)≧30% CD38⁺ cells (CR+PR-high; n=11 (58%)) (FIG. 5B). Whereas, all the patients in the SD+PD group had ≧30% CD38⁺ cells (SD+PD-high; n=11 (100%)). This difference was found to be statistically significant (p=0.014; Fisher's Exact test). However, when the groups were compared at screening ignoring the low/high threshold, there was no difference in the response groups (p-value=0.1032). In the CR+PR-low group, there was a significant increase in the percentage of CD38⁺ cells at week 9 compared to screen (p=0.0234; Wilcoxon Signed Rank test) (FIG. 5C). This increase was seen, on average, as early as week 1 and lasted until week 11, which was the last time point of assessment for CD38⁺ cells (data not shown). No significant difference was seen in the percentage of CD38⁺ cells between screen and week 9 in CR+PR-high (p=0.5469; Wilcoxon Signed Rank test) or SD+PD-high (p=1.0; Wilcoxon Signed Rank test) groups (FIG. 5C). There was no significant difference in the percentage of CD38⁺ cells at week 9 among CR+PR-low, CR+PR-high, and SD+PD-high groups (p=0.4083; Wilcoxon Rank Sum test). These results indicate that L+FCR treatment is effective in treating patients with high CD38 expression.

### Example 5

### Lumiliximab Combination Treatment is Effective Independent of β2-Microglobulin Levels

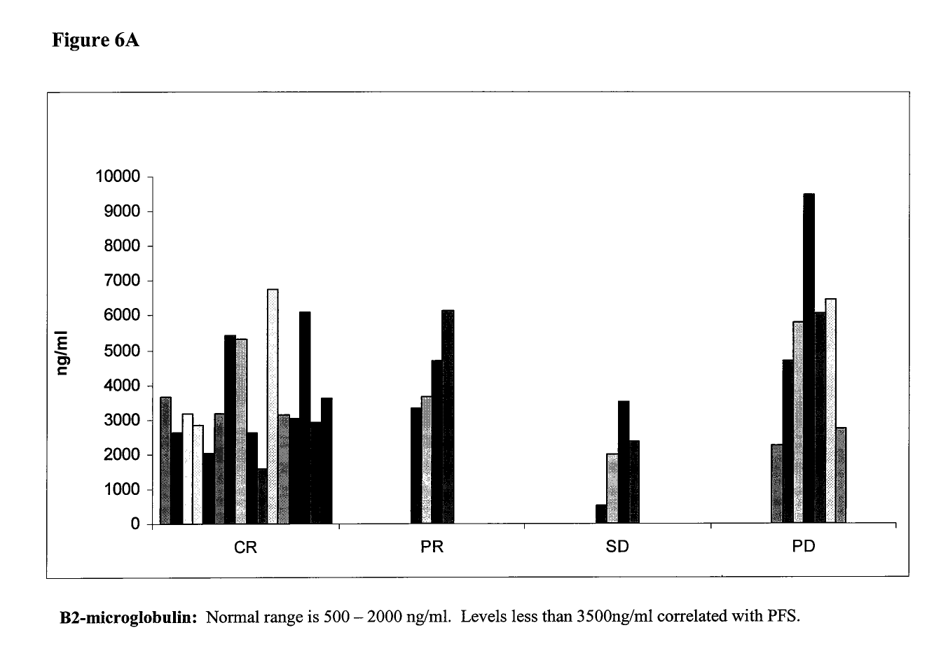

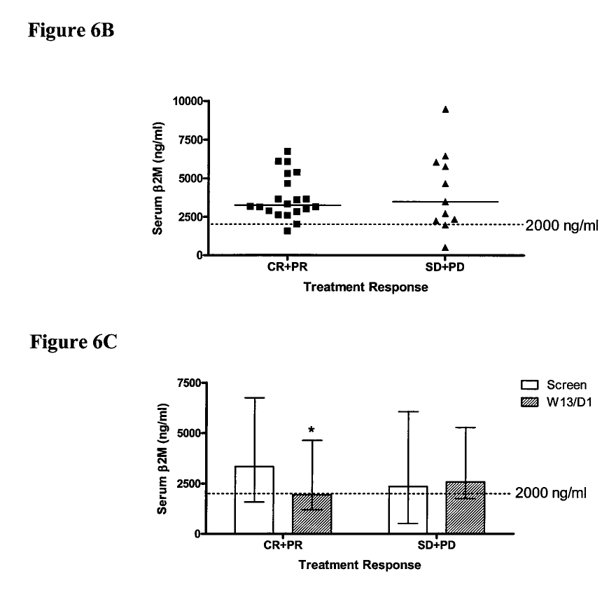

Similarly, L+FCR was effective in treating patients independent of β2-microglobulin levels. Patients were treated with L+FCR as described in Example 2. Normal β2-microglobulin levels are 500-2000 ng/ml, and levels of less than 3500 ng/ml are correlated with progression free survival (PFS). β2-microglobulin levels were assessed using ELISA and some patients with both high and low levels of β2-microglobulin responded completely or partially to L+FCR treatment. See FIG. 6A.

The level of β2M at screen was not significantly different in patients in the CR+PR group compared with those in the SD+PD group (p=0.95; Wilcoxon Rank Sum test) (FIG. 6B). Most (28 out of 31) of the patients had β32M over 2000 ng/mL, which is considered to be a poor prognostic factor in the early stages of disease. At week 13, there was a significant decrease in the β2M levels in patients in the CR+PR group compared to screen (p<0.0001; Wilcoxon Signed Rank test) (FIG. 6C). On the other hand, no significant difference was found in the β2M levels pre-treatment and at week 13 in patients in the SD+PD group (p=0.625; Wilcoxon Signed Rank test). Out of 17 patients in the CR+PR group who had β2M levels recorded at both the visits, 16 patients (94%) had at least a 25% decrease (average decline 40%) in the β2M levels at week 13 from screen; whereas, only 2 patients out of 5 (40%) had at least a 25% decrease in the P2M levels at week 13 from screen in the SD+PD group. This difference was statistically significant (p=0.0239; Fisher's Exact test).

### Example 6

### Lumiliximab Combination Treatment is Effective Independent of Pre-Treatment Levels of Soluble CD23

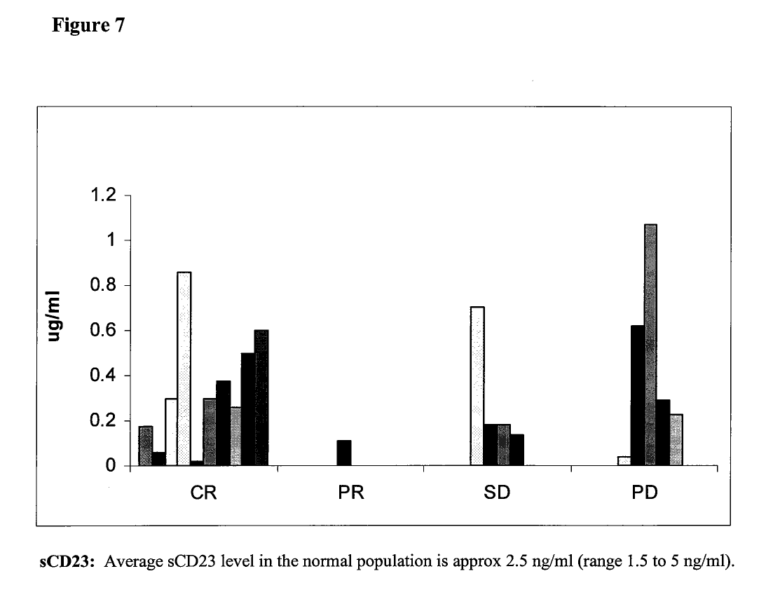

Soluble CD23 is another marker for poor prognosis in CLL patients. Sarfati et al. Blood 88: 4259 (1996). In general, CLL patients have higher levels of soluble CD23 than the average population. The average soluble CD23 levels range from about 1.5 to about 5 ng/ml. In contrast, CLL patients frequently have higher levels. In addition, among CLL patients, higher levels of soluble CD23 have been associated with poor prognosis. CLL patients with varying levels of soluble CD23 (as measured by ELISA) were treated with L+FCR as described in Example 2, and pretreatment levels of soluble CD23 were not correlated with efficacy of L+FCR treatment. See FIG. 7. Therefore, lumiliximab combination treatment is effective independent of pre-treatment soluble CD23 levels.

### Example 7

### Lumiliximab Combination Treatment is Effective Independent of CD23 Expression Intensity

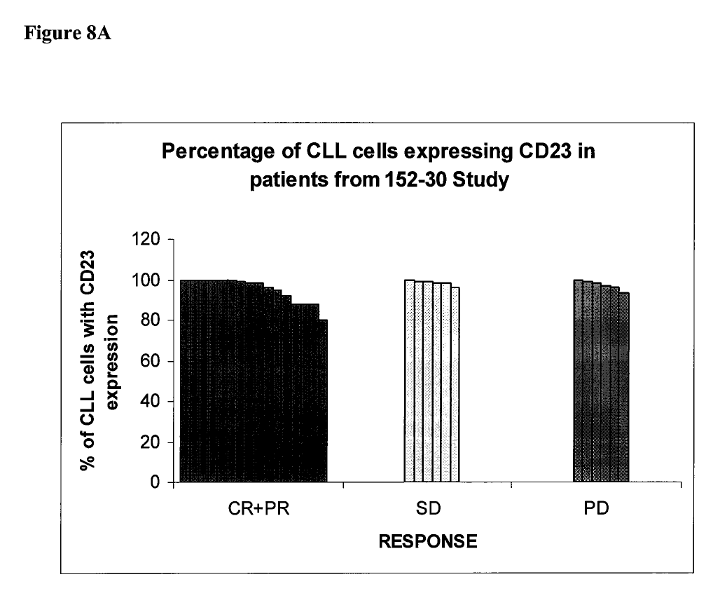

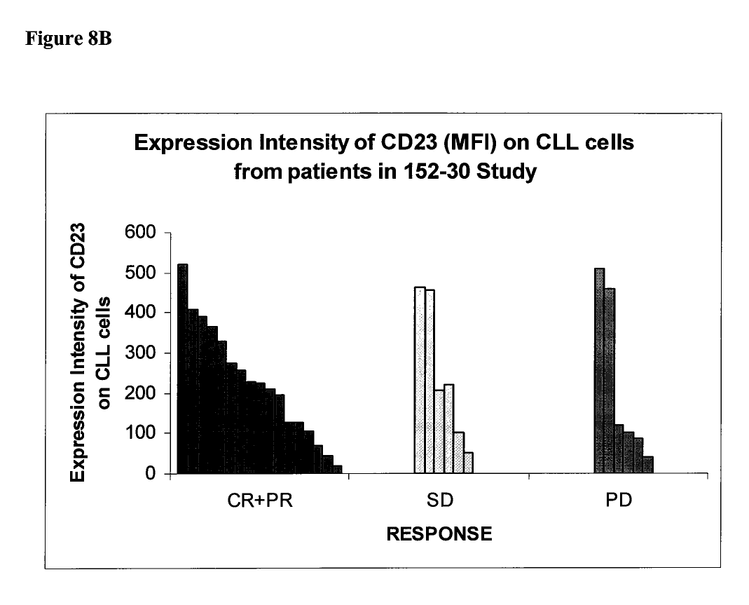

The effect of CD23 expression levels on efficacy of L+FCR treatment was also evaluated. Assessment of the expression of CD23 in CLL cells using ELISA demonstrated that the vast majority of CLL cells express CD23 (see FIG. 8A), but the expression intensity varied between patients (see FIG. 8B). However, patients' responses to treatment with L+FCR as described in Example 1 were independent of CD23 expression intensities. These results suggest that lumiliximab combination treatment is effective independent of CD23 expression intensity.

### Example 8

### IgV_{H} Mutation Status

A reasonable homology cut-off of 98% has been confirmed by many groups to distinguish patients with “mutated” versus “unmutated” IgV_{H}. Out of 16 patients with IgV_{H} status analyzed in the CR+PR group, 12 patients (75%) had unmutated IgV_{H} status, classifying them into poor prognosis category, while 91% of patients (10 out of 11) had unmutated IgV_{H} in the SD+PD group. This difference was not statistically significant (p=0.6185; Fisher's Exact test).

### Example 9

### Association Between Rituximab Resistance Markers (CD55 and CD59) with Treatment Outcome in 152-30 Study

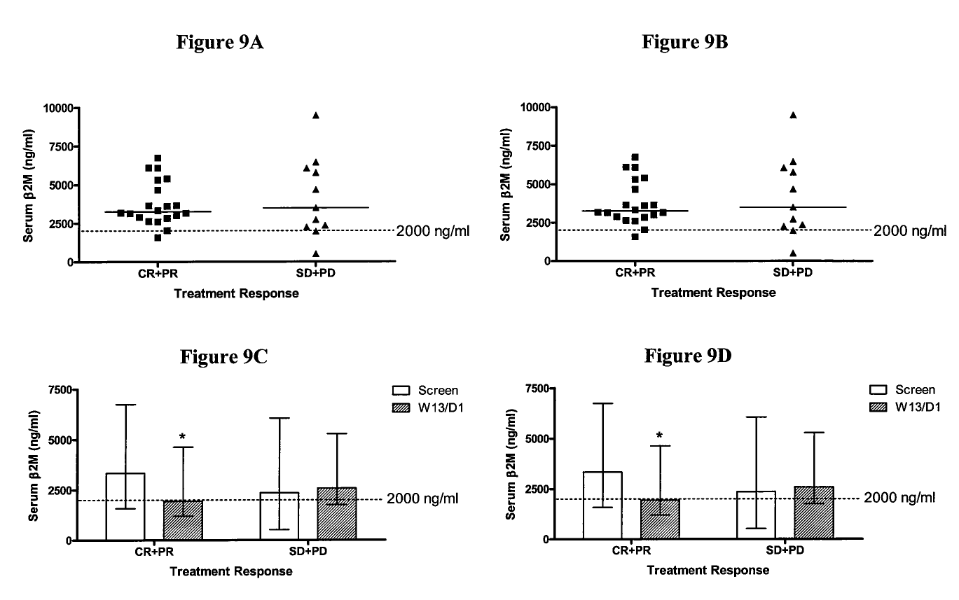

The level of CD55 expression on CLL cells at screen was not significantly different in patients in the CR+PR group compared to those in the SD+PD group (p=1.0; Wilcoxon Rank Sum test) (FIG. 9A). No significant difference was seen in CD55 expression levels at least one cycle after treatment (average of week 5 and week 9) compared to screen in either of the groups (p=0.5949, CR+PR and p=0.2783, SD+PD; Wilcoxon Signed Rank test) (FIG. 9B).

There was a trend towards a higher CD59 expression at screen in the SD+PD group compared to CR+PR, but this difference was not statistically significant (p=0.0523; Wilcoxon Rank Sum test) (FIG. 9C). Interestingly, after 1 cycle of treatment (average of week 5 and week 9), CD59 expression increased significantly compared to screen in only the CR+PR group (p=0.0001; Wilcoxon Signed Rank test) and not in the SD+PD group (p=0.1426; Wilcoxon Signed Rank test) (FIG. 9D). However, percentage of patients having at least 2-fold increase in CD59 expression after at least 1 cycle compared to screen was not significantly different between CR+PR (53%) and SD+PD (18%) groups (p=0.1213; Fisher's Exact test), probably due to a smaller sample size.

### Example 10

### Lumiliximab Induced Downmodulation of Anti-Apoptotic Proteins

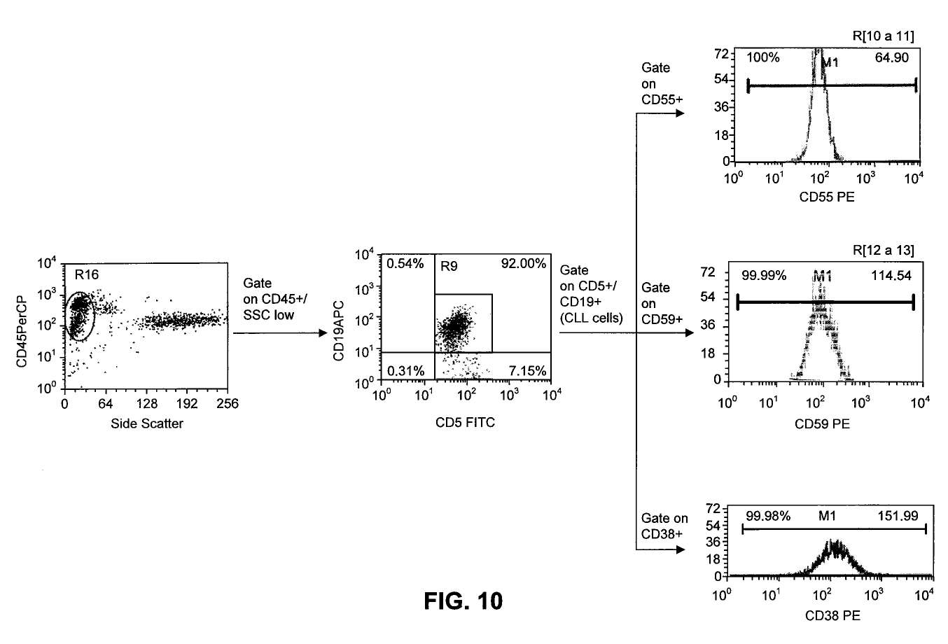

Patients can develop resistance to therapies with CLL cells exhibiting an apoptosis-resistant phenotype due to enhanced survival signaling via overexpression of anti-apoptotic proteins such as Bcl-2, Mcl-1 and XIAP. The effect of lumiliximab treatment on levels of anti-apopotic proteins was measured. FIG. 10A shows that the levels of Mcl-1, Bcl-XL, Bcl-2 were downregulated after lumilixumab treatment, whereas Bad, Bid, and β-actin control were not. FIG. 10B shows that the levels of XLAP were downregulated, whereas levels of cIAP-1, cLAP-2, survivin, and β-actin control were not. In addition, pre- and post-treatment patient samples obtained from Phase I/II lumiliximab monotherapy trial in relapsed CLL showed activation of caspase-3, along with downregulation of Bcl-2 and Mcl-1.

1. A method of treating a CD23+ malignancy in a mammal in need thereof comprising administering to said mammal a therapeutic amount of a CD23 antibody or fragment thereof, wherein the mammal over expresses a poor prognostic marker.

2. A method of treating a leukemia in a mammal in need thereof comprising administering to said mammal a therapeutic amount of a CD23 antibody or fragment thereof, wherein the mammal over expresses a poor prognostic marker.

3. A method of treating a CD23+ malignancy in a mammal in need thereof comprising (1) assessing whether said mammal overexpresses a poor prognostic marker; and (2) administering to said mammal a therapeutic amount of a CD23 antibody or fragment thereof if said mammal overexpresses said poor prognostic marker.

4. A method of treating a leukemia in a mammal in need thereof comprising (1) assessing whether said mammal overexpresses a poor prognostic marker; and (2) administering to said mammal a therapeutic amount of a CD23 antibody or fragment thereof if said mammal overexpresses said poor prognostic marker.

5. A method of designing a chemotherapeutic regimen comprising (1) assessing whether said mammal overexpresses a poor prognostic marker; and (2) administering to said mammal a therapeutic amount of a CD23 antibody or fragment thereof depending on the expression level of said poor prognostic marker.

6. The method of claim 3 wherein said mammal has been demonstrated to overexpress said poor prognostic marker.

7. The method of claim 1, wherein said method further comprises determining whether said mammal over expresses said poor prognostic marker.

8. A method of inducing apoptosis in a cell expressing a poor prognostic marker comprising contacting the cell with a CD23 antibody.

9. The method of claim 3, wherein the poor prognostic marker is selected from the group ZAP70, CD38, and β2-microglobulin.

10. The method of claim 3, wherein the CD23 antibody is lumiliximab, an antigen binding fragment thereof, an antibody that competitively inhibits binding of lumiliximab to CD23 or an antigen binding fragment thereof.

11. The method of claim 10, wherein the CD23 antibody is lumiliximab.

12. The method of claim 3 wherein said administration results in increased caspase-3 activity.

13. The method of claim 3, wherein said administration results in increased apoptosis of malignant cells.

14. The method of claim 3 wherein said mammal is human.

15. The method of claim 3 wherein administration of said CD23 antibody is achieved by oral administration, nasal administration, parenteral administration, transdermal administration, topical administration, intraocular administration, intrabronchial administration, intraperitoneal administration, intravenous administration, subcutaneous administration, intramuscular administration, buccal administration, sublingual administration, vaginal administration, by inhalation, by an implanted pump, and a combination of two or more thereof.

16. The method of claim 15 wherein said administration is achieved via intravenous administration.

17. The method of claim 3 further comprising administering at least one additional pharmaceutical compound effective for treating, preventing or inhibiting a malignancy.

18. The method of claim 17 wherein said at least one additional pharmaceutical compound is selected from the group consisting of therapeutic antibodies, immunosuprresive agents, cytotoxic agents, chemotherapeutic agents and/or cytokines.

19. The method of claim 3 further comprising administering fludarabine, cyclophosphamide and rituximab.

20. The method of claim 3 wherein said CD23+ malignancy is selected from the group consisting of relapsed Hodgkin's disease, resistant Hodgkin's disease high grade, low grade and intermediate grade non-Hodgkin's lymphoma, B cell chronic lymphocytic leukemia (B-CLL OR CLL), lymhoplasmacytoid lymphoma (LPL), mantle cell lymphoma (MCL), follicular lymphoma (FL), diffuse large cell lymphoma (DLCL), Burkitt's lymphoma (BL), AIDS-related lymphoma, monocytic B cell lymphoma, angioimmunoblastic lymphoadenopathy, small lymphocytic; follicular, diffuse large cell; diffuse small cleaved cell; large cell immunoblastic lymphoblastoma; small, non-cleaved; Burkitt's and non-Burkitt's; follicular, predominantly large cell, follicular, predominantly small cleaved cell; and follicular, mixed small cleaved and large cell lymphomas.

21. The method of claim 20 wherein the CD23+ malignancy is B cell chronic lymphocytic leukemia (CLL).

22. The method of claim 4 wherein the leukemia is B cell chronic lymphocytic leukemia (CLL).

23. The method of claim 21 wherein the poor prognostic marker is ZAP70 or CD38 and wherein the poor prognostic marker is expressed on 30% or more of the CLL cells of the mammal.

24. The method of claim 23 wherein the poor prognostic marker is expressed on 50% or more of the CLL cells of the mammal.

25. The method of claim 24 wherein the poor prognostic marker is expressed on 70% or more of the CLL cells of the mammal.

26. The method of claim 21 wherein the poor prognostic marker is β2 microglobulin, and wherein β2 microglobulin levels are greater than 3500 ng/ml.

27. The method of claim 26 wherein the β2 microglobulin levels are greater than 5000 ng/ml.

28. The method of claim 21 wherein the poor prognostic marker is soluble CD23 and wherein soluble CD23 levels are greater than 100 μg/ml.

29. The method of claim 28, wherein soluble CD23 levels are greater than 200 μg/ml.

30. The method of claim 28, wherein soluble CD23 levels are greater than 300 μg/ml.
